(19) United States
(12) Patent Application Publication
(10) Pub. No.: US 2013/0211281 A1
(43) Pub. Date: Aug. 15, 2013

(54) SENSOR SYSTEM FOR MONITORING A FOOT DURING TREATMENT AND REHABILITATION

(71) Applicant: MedHab, LLC, (US)

(72) Inventors: Johnny Ross, Mansfield, TX (US); Jagdeepinder Singh Sanghera, Mansfield, TX (US)

(73) Assignee: MEDHAB, LLC, Mansfield, TX (US)

(21) Appl. No.: 13/741,294

(22) Filed: Jan. 14, 2013

Related U.S. Application Data
(63) Continuation-in-part of application No. 13/070,649, filed on Mar. 24, 2011, now Pat. No. 8,384,551.
(60) Provisional application No. 61/585,891, filed on Jan. 12, 2012.

(51) Int. Cl. A61B 5/01; A61B 5/00; G01K 13/00

(52) U.S. Cl. CPC A61B 5/01; G01K 13/002; A61B 5/6807
USPC 600/549

(57) ABSTRACT

A sensor system includes a temperature sensor array that includes a plurality of temperature sensors, and a microcontroller operably connected with the temperature sensor array and an antenna for transmitting data received from the temperature sensor array. A data collection system receives the data transmitted from the microcontroller and stores it in a database operably installed in the memory of the data collection system for storing the data. A user interface program displays the data in a format suitable for determining if there is a problem with the foot based upon changes in the temperature of the foot.

### CROSS-REFERENCE TO RELATED APPLICATIONS

This application for a utility patent is a continuation-in-part of a previously filed utility patent, still pending, having application Ser. No. 13/070,649, filed Mar. 24, 2011. This application for a utility patent also claims the benefit of U.S. Provisional Application No. 61/585,891, filed Jan. 12, 2012.

### STATEMENT REGARDING FEDERALLY SPONSORED RESEARCH

Not Applicable

### BACKGROUND OF THE INVENTION

1. Field of the Invention

This invention relates generally to sensor systems for monitoring a person's foot, and more particularly to a sensor system adapted to be worn on a foot for monitoring temperature changes in a foot during treatment or rehabilitation of the foot.

2. Description of Related Art

There are various devices in the prior art that teach sensor devices for measuring pressure placed upon a user's foot for the purposes of assisting in rehabilitation of a user's leg following an injury. Knotts et al., U.S. Pat. No. 5,107,854, for example, teaches a slipper that includes a fluid chamber that enables weight sensing by a load monitor. When not enough weight is applied, or when too much weight is applied, a beeping sound is emitted to guide the patient in rehabilitating an injured leg.

Other examples of such devices include Huberti, U.S. Pat. No. 5,042,504, which teaches an insertable sole that includes plates having force sensors for determining a load placed upon the sole by a user; Gray, U.S. Pat. No. 5,269,081, which teaches a force monitoring shoe that includes a spring, a sensor for sensing force applied to the spring, and a feedback mechanism that may include a beeper, flashing LEDs, a shocking element, and vibrational feedback; Gray, U.S. Pat. No. 5,357,696; Schmidt et al., U.S. Pat. No. 5,619,186; Schmidt et al., U.S. Pat. No. 5,408,873; Avni et al, U.S. Pat. No. 6,273,863; Fullen et al., U.S. Pat. No. 5,323,650; and Gray, U.S. Pat. No. 6,122,846.

There are also various sensor devices that include accelerometers for various purposes. For example, Hirsch et al., U.S. Pat. No. 6,578,291, teaches a shoe having a built-in electronic wear indicator device that includes an accelerometer for measuring foot movement; Damen et al., U.S. Pat. No. 6,356,856, teaches a system built into a shoe or measuring the speed of a person while running or walking; and similar shoes are also shown in Huang, U.S. Pat. No. 5,875,571, Huang, U.S. Pat. No. 5,815,954, Hutchings, U.S. Pat. No. 5,724,265, and Huang, U.S. Pat. No. 5,661,916.

The above-described references are hereby incorporated by reference in full.

The prior art teaches various forms of sensors adapted to be worn on the feet of a user for tracking movement of the foot, and for sensing a temperature of the foot. However, the prior art does not teach a sensor device that includes a sensor array that is particularly adapted for monitoring the temperature of the foot at multiple critical locations to help prevent foot infections, ulcers, restricted blood flow, and the like, particularly in diabetic patients. The prior art also does not teach the present method of monitoring the user's foot temperature to most accurately detect foot ulcers or restricted blood flow at an early stage to enable quick preventative treatment and to avoid more serious problems that might threaten serious injury to the patient. The present invention fulfills these needs and provides further advantages as described in the following summary.

### SUMMARY OF THE INVENTION

The present invention teaches certain benefits in construction and use which give rise to the objectives described below.

The present invention provides a sensor system for monitoring a foot. The sensor system includes a temperature sensor array that includes a plurality of temperature sensors disposed on a substrate layer, and a microcontroller operably connected with the temperature sensor array and an antenna for transmitting data received from the temperature sensor array. A data collection system receives the data transmitted from the microcontroller and stores it in a database operably installed in the memory of the data collection system for storing the data. A user interface program displays the data in a format suitable for determining if there is a problem with the foot based upon changes in the temperature of the foot.

A primary objective of the present invention is to provide a sensor system having advantages not taught by the prior art.

Another objective is to provide a sensor system that includes a sensor device having a temperature sensor array that is particularly adapted for monitoring the temperature of the foot at multiple critical locations to help prevent foot infections, ulcers, restricted blood flow, and the like, particularly in diabetic patients.

Another objective is to provide a sensor system that includes an inductive charging mechanism so that the sensor system remains powered and ready for use, so that critical data is not missed at suitable measurement times.

A further objective is to provide a method of monitoring the user's foot temperature to most accurately detect foot ulcers or restricted blood flow at an early stage to enable quick preventative treatment and to avoid more serious problems that might threaten serious injury to the patient.

A further objective is to provide a method of monitoring the user's foot temperature at a suitable predetermined time so that outside variables do not unduly interfere with temperature measurements.

Other features and advantages of the present invention will become apparent from the following more detailed description, taken in conjunction with the accompanying drawings, which illustrate, by way of example, the principles of the invention.

### BRIEF DESCRIPTION OF THE DRAWING

The accompanying drawings illustrate the present invention. In such drawings:

FIG. 1 is a block diagram of a sensor system according to one embodiment of the present invention, wherein the sensor system includes a pair of sensor devices in the form of insoles, and a data collector device;

FIG. 2 is a top plan view of one of the sensor devices of FIG. 1, the sensor device including a temperature sensor array utilized in one embodiment of the sensor system;

FIG. 3A is a side perspective view of a slipper that incorporates the sensor device of FIG. 2;

FIG. 3B is close-up side perspective of a sole of the slipper of FIG. 3A, illustrating inner and outer layers of the sole;

FIG. 4 is a perspective view of a thin film resistive thermal device utilized in one embodiment of the temperature sensor array of FIG. 2;

FIG. 5 is a schematic of one example of the resistive thermal device;

FIG. 6 is a perspective view of a thermocouple used in another embodiment of the sensor system;

FIG. 7 is a block diagram of another embodiment of the sensor system of FIG. 1, illustrating a network embodiment thereof; and

FIG. 8 is an exploded perspective view of yet another embodiment of the sensor device;

FIG. 9 is a perspective view thereof once the sensor device has been assembled;

FIG. 10 is a sectional view thereof along lines 10-10 in FIG. 9;

FIG. 11 is a perspective view of an inductive charging station used to charge the insoles; and

FIG. 12 is a block diagram an alternative embodiment of the sensor system, wherein the insoles communicate with a portable electronic device such as a cell phone.

### DETAILED DESCRIPTION OF THE INVENTION

The above-described drawing figures illustrate the invention, a sensor system 10 for monitoring a foot during treatment and rehabilitation.

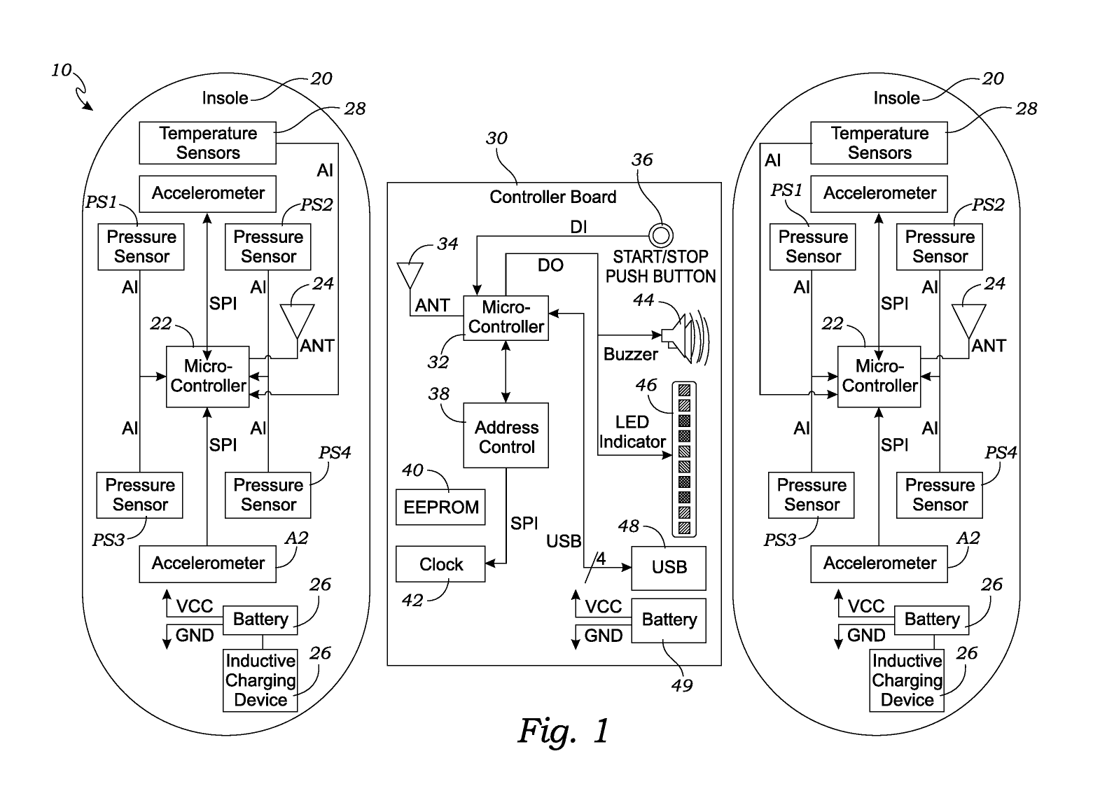

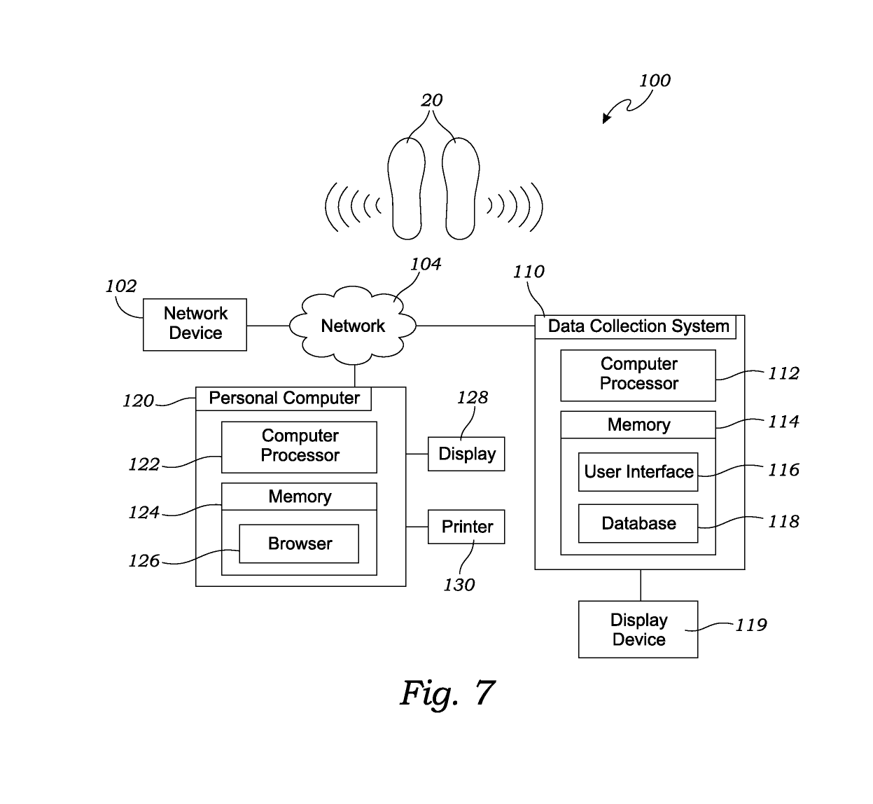

FIG. 1 is a block diagram of the sensor system 10 according to one embodiment of the present invention. As illustrated in FIG. 1, in this embodiment the sensor system 10 includes a pair of sensor devices 20 in the form of insoles, and a data collection system 30 for receiving data from the insoles 20. In this embodiment, the data collection system 30 is a data collector device that remains proximate to the user; however, alternative data collection systems may also be utilized, including different forms of devices, and as a remote data collection system 110 illustrated in FIG. 7 and discussed in greater detail below. The data may be received, collected, reviewed, and utilized using different forms of computer devices and systems, as discussed in greater detail below.

Furthermore, while the insoles 20 are illustrated as a single pair, this is hereby defined to include the use of a single insole when warranted, or multiple insoles or pairs of insoles, depending upon the particular implementation of the invention.

As illustrated in FIG. 1, each of the sensor devices 20 may include pressure sensors PS1-PS4 and accelerometers A1-A2 (collectively “the sensors”) for measuring pressures against the insole 20 and movement of the insole 20. Several embodiments of these elements are discussed below, and various arrangement of the sensor may be used. As illustrated in FIG. 1, in one embodiment the sensors may be operably connected with a microcontroller 22 (e.g., RF uC), which is operably connected with an antenna 24 for transmitting data received from the sensors. The microcontroller 22 may also contain, or be operably attached with, a memory (not shown) for storing the data for later transmission.

As illustrated in FIG. 1, the sensor device 20 of this embodiment may further include one or more temperature sensors 28 for also sensing the temperature of the user's foot. Such temperature measurements may be taken in conjunction with movement and pressure data, or independently thereof. Several embodiments of the temperature sensors 28 are discussed in greater detail below; however, those skilled in the art may devise alternative embodiments, and such alternatives (and equivalents thereof) should be considered within the scope of the present invention. The microcontroller 22 is operably connected to a battery 26 (defined to include any form of suitable power source). The battery 26 may be rechargeable, and in this embodiment is rechargeable via an inductive charging device 26, as discussed in greater detail below.

As illustrated in FIG. 1, the data collector device 30 of this embodiment may include a microcontroller 32 also operably connected with an antenna 34 for receiving data received from the insoles 20. The data collector device 30 may further include a start/stop pushbutton 36 for turning the unit on/off, and/or otherwise triggering the function of the sensor device 20. For purposes of this application, the term pushbutton is hereby defined to include any form or switch, button, voice activation, and/or equivalent switch or other control mechanism for turning on, actuating, triggering, or otherwise operating the sensor system 10.

The microcontroller 32 may be operably connected (e.g., SPI) through an address control 38 to a suitable memory device 40 (e.g., EEPROM) and a clock 42 for handling data from the sensor device 20. Various outputs may also be included, including a buzzer 44 for reporting various events (start, stop, warning, parameters of a rehabilitation exercise, etc.), and/or an LED indicator 46 for similarly reporting various events, and/or any number of similar or equivalent reporting mechanisms known in the art.

The microcontroller 32 is operably connected to a battery 49, and may also be operably connected to an interface mechanism 48 (e.g., a USB port, Bluetooth, etc.) for operably connecting with an outside computer for reporting data, receiving software updates, etc. This connection may also be made wirelessly, using systems known in the art and therefore not discussed in greater detail herein. The battery 49 may be rechargeable, or easily replaceable, and may also be rechargeable using induction, as described below.

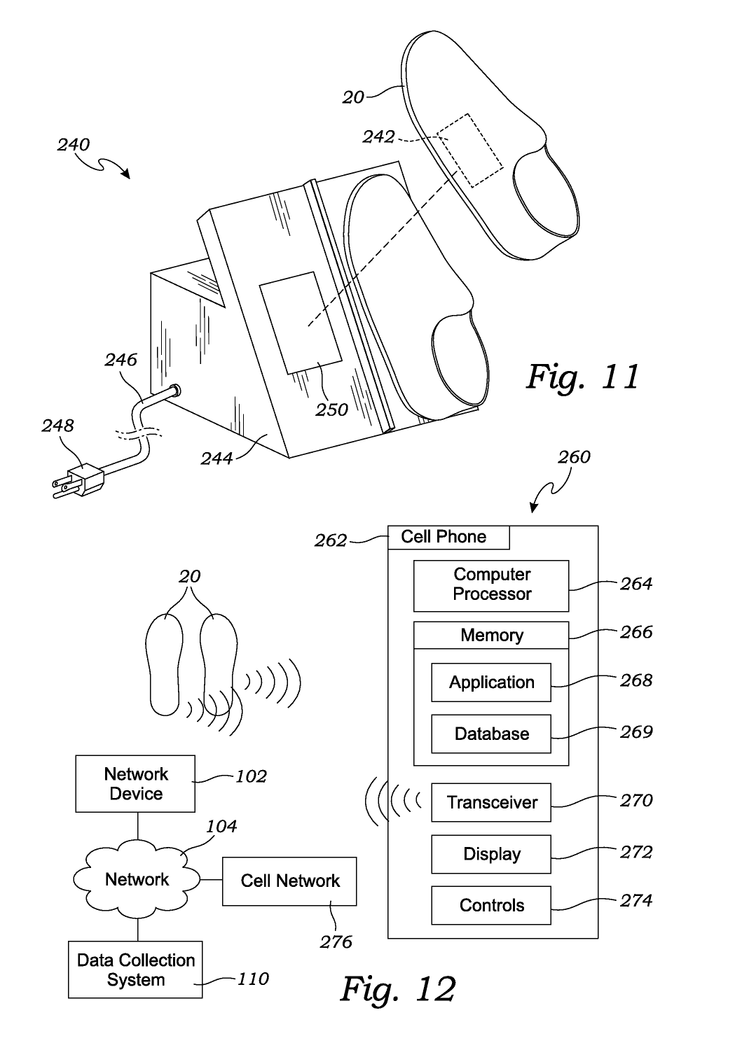

While one embodiment of the data collector device 30 is illustrated herein, those skilled in the art may devise alternative devices for receiving the data, and such alternatives should be considered within the scope of the present invention. In one embodiment, illustrated in FIG. 11 and discussed in greater detail below, the data collector device 30 may be a cell phone, tablet computer, or other similar form of portable electronic device, with the necessary functionality being provided by a downloadable software application (commonly called an “app”).

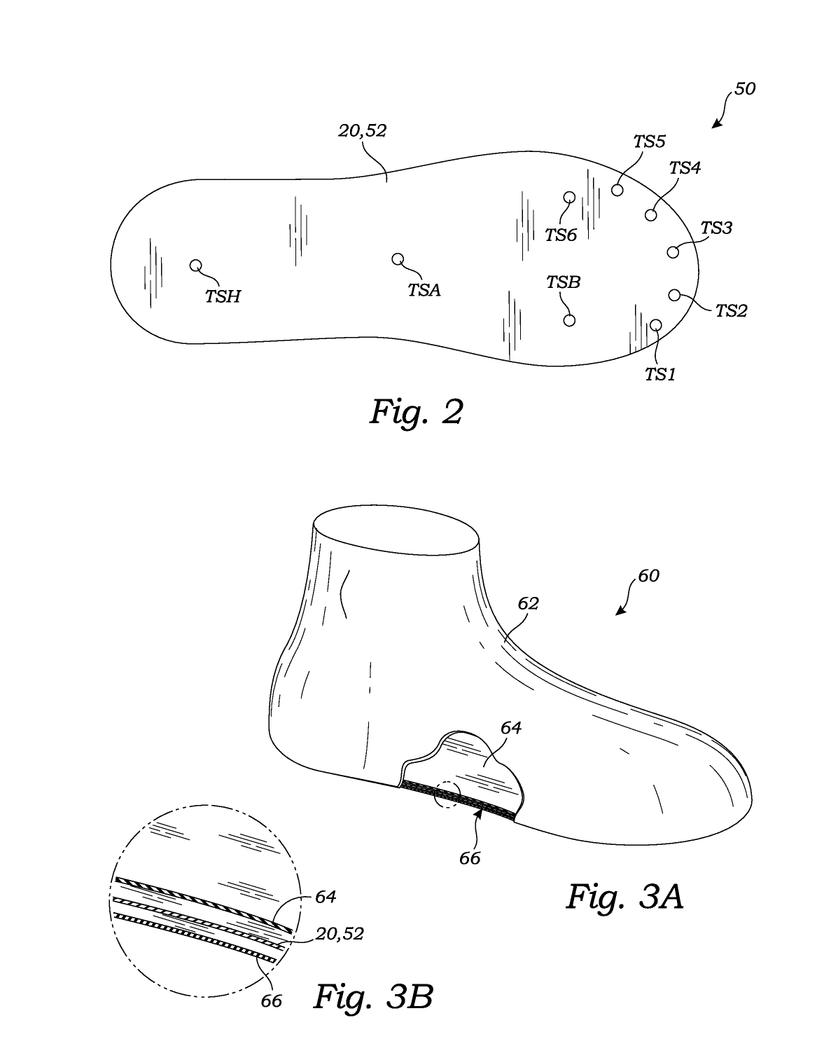

FIG. 2 is a top plan view of one of the sensor devices 20 of FIG. 1, illustrating one embodiment of a temperature sensor array 50 utilized in one embodiment of the sensor system 10. In the embodiment of FIG. 2, the temperature sensor array 50 includes a plurality of temperature sensors TS1-TS6 (and optionally TSB, TSA, and TSH, discussed below, as well), disposed on a suitable substrate layer 52. In this case five toe temperature sensors TS1-TS5 are disposed on the substrate layer 52 to contact each of the user's toes, and a sixth sensor, a pad sensor TS6, is disposed to contact a pad of the user's foot laterally spaced from a ball of the user's foot. Such an arrangement is particularly well suited for measuring the temperature in each of the user's toes, which may be beneficial in treating diabetes, which can often impair blood flow in the toes.

Additional temperature sensors may further include an arch temperature sensor TSA that is positioned on the substrate 52 to operably contact an arch of the user's foot; and a heel temperature sensor TSH that is operably positioned on the substrate 52 to contact a heel of the user's foot. While FIG. 2 illustrates one embodiment of how the temperature sensors may be arrayed, those skilled in the art may devise alternative arrangements, and such alternatives should be considered within the scope of the present invention.

FIG. 3A is a side perspective view of a slipper 60 (i.e., footwear, sock, or other suitable element) that incorporates the sensor device 20 of FIG. 2. A portion of the illustration is shown broken away to better illustrate the construction of the slipper 60. In this embodiment, the slipper 60 includes an upper 62 for holding the slipper 60 on the user's foot.

FIG. 3B is close-up side perspective of a sole of the slipper 60 of FIG. 3A, illustrating inner and outer layers 64 and 66 of the sole. In this embodiment, the inner layer 64 is adapted for contacting the user's foot, and an outer layer 66 is adapted for contacting the ground. The inner layer 64 is preferably padded and otherwise suitable for being worn by the user, and the outer layer 66 is preferably more rigid and durable for providing good wear against the ground. The sensor device 20 (or the substrate 52 portion thereof) may be positioned between the inner and outer layers 64 and 66. Various embodiments of the sensor device 20 are illustrated herein, and any form of sensor device 20 may be utilized in this embodiment of the invention. Furthermore, while the slipper 60 may include the upper 62 illustrated, other embodiment of suitable footwear may be used (e.g., any form of suitable shoes, boots, booties, socks, sandals, etc.).

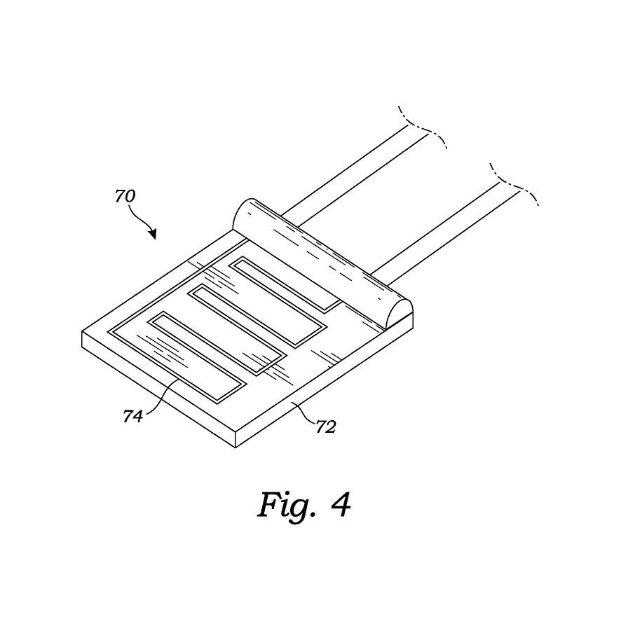

FIG. 4 is a perspective view of a thin film resistive thermal device 70 utilized in one embodiment of the temperature sensor array 50 of FIG. 2. In this embodiment, the temperature sensors are formed by the thin film resistive thermal device 70 that includes a thin substrate 72 formed of a suitable material (e.g., ceramic, or other suitable material known in the art). A resistive material 74 (e.g., platinum, etc.) is etched or otherwise formed on the substrate 72 in a suitable pattern using techniques known in the art. A sealing layer (not shown) may be included to protect the resistive material 74 (e.g., epoxy, etc.), although this is not required, and may be omitted in some embodiments.

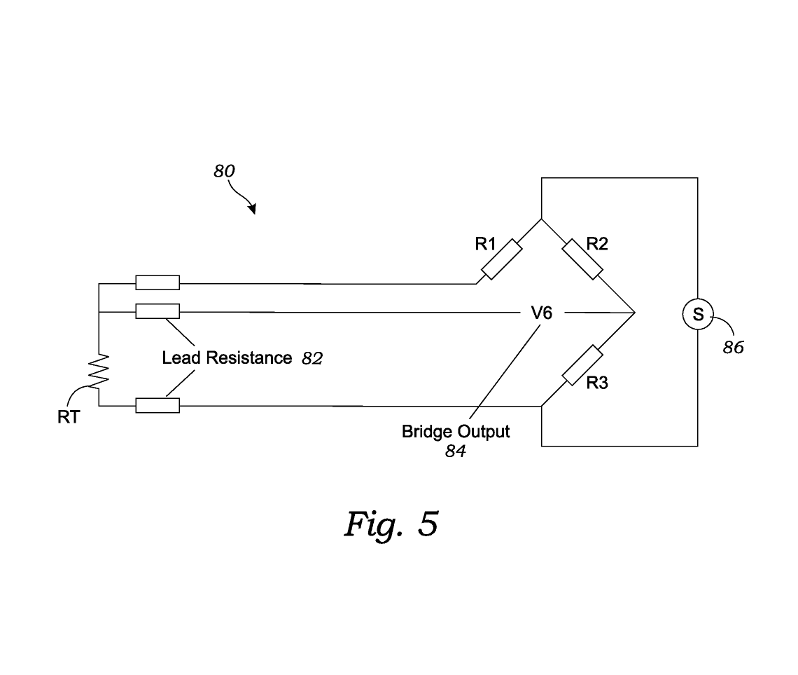

FIG. 5 is a schematic of one example of the resistive thermal device 80. In this embodiment, a resistance element RT is operably connected through lead resistance 82 to resistors R1, R2, and R3, which are operably connected to a power source 86 and to a bridge output 84. While this one embodiment is illustrated, those skilled in the art may utilize alternative circuits, and such alternatives should be considered within the scope of the present invention.

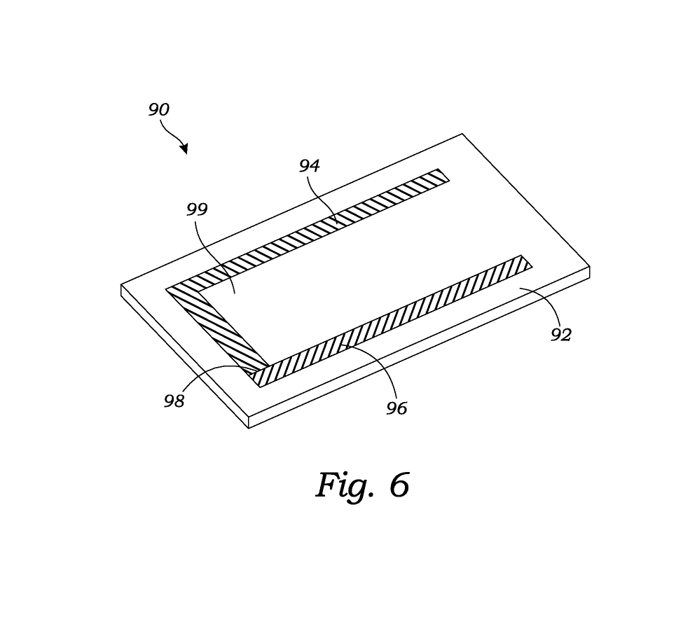

FIG. 6 is a perspective view of a thermocouple 90 used in another embodiment of the sensor device 20 of FIG. 1. In this embodiment, the thermocouple 90 includes a substrate 92 that includes a first leg 94 and a second leg 96 that meet at a junction 98. A spacer 99 of the substrate 92 separates the first and second legs 94 and 96. The first leg 94 is constructed of a different metal than the second leg 96, using pairs of metals that are suitable for measuring temperatures using techniques well known in the art via the interaction of the legs 94 and 96 at the junction 98.

FIG. 7 is a block diagram of another embodiment of a sensor system 100 that includes the sensor devices 20 (i.e., insoles) of FIG. 1, or alternative embodiments thereof, and a data collection system 110. In the embodiment of FIG. 7, the insoles 20 communicate via a network device 102 and a network 104 (e.g., the Internet) with the data collection system 110 with a that enables remote data collection, and reporting of the data to a variety of users (e.g., doctors, physical therapists, the user, and other associated professionals and caregivers). In the embodiment of FIG. 7, the data collection system 110 includes a computer that includes a computer processor 112, a memory 114, and a display device 119 (e.g., a monitor, printer, or other form of output). The computer 110 may be located remotely, typically in a central location that may serve a large number of users. In alternative embodiments, however, the data may be collected and stored locally, such as in a portable electronic device or personal computer.

The computer processor 112 and the memory 114 may be any form of processor(s), memory device(s), and/or microprocessor combination known in the art. The memory 114 of the data collection system 110 contains software to implement the system, store the data, and report results. In the present embodiment, the data collection system 110 includes a user interface 116 for enabling users to review the data collected, and a database 118 for storing the data.

In addition to the display device 119 for displaying results at a specific location, the data collection system 110 is adapted to be used in conjunction with external devices, such as a personal computer 120 that accesses the data collection system 110 via the network 104. The term “personal computer” is hereby defined to include similar computer devices (e.g., smart phones, tablet computers, etc.). The personal computer 120 include a computer processor 122 and memory 124, as discussed above. The memory 124 may include a browser 126 or similar program for accessing the data stored in the data collection system 110. The browser 126 may be a general use program (e.g., Internet Explorer, Firefox, etc.) or a proprietary program adapted particularly for operation in conjunction with the data collection system 110. If it is a proprietary program, additional security and encryption may be included for added security of the data.

The personal computer 120 may include a display 128 for displaying the data, and/or a printer 130 for enabling data to be printed and analyzed, and/or any other peripheral devices known in the art. Since the general construction of these computer devices and peripherals are well known in the art, they are not described in greater detail herein.

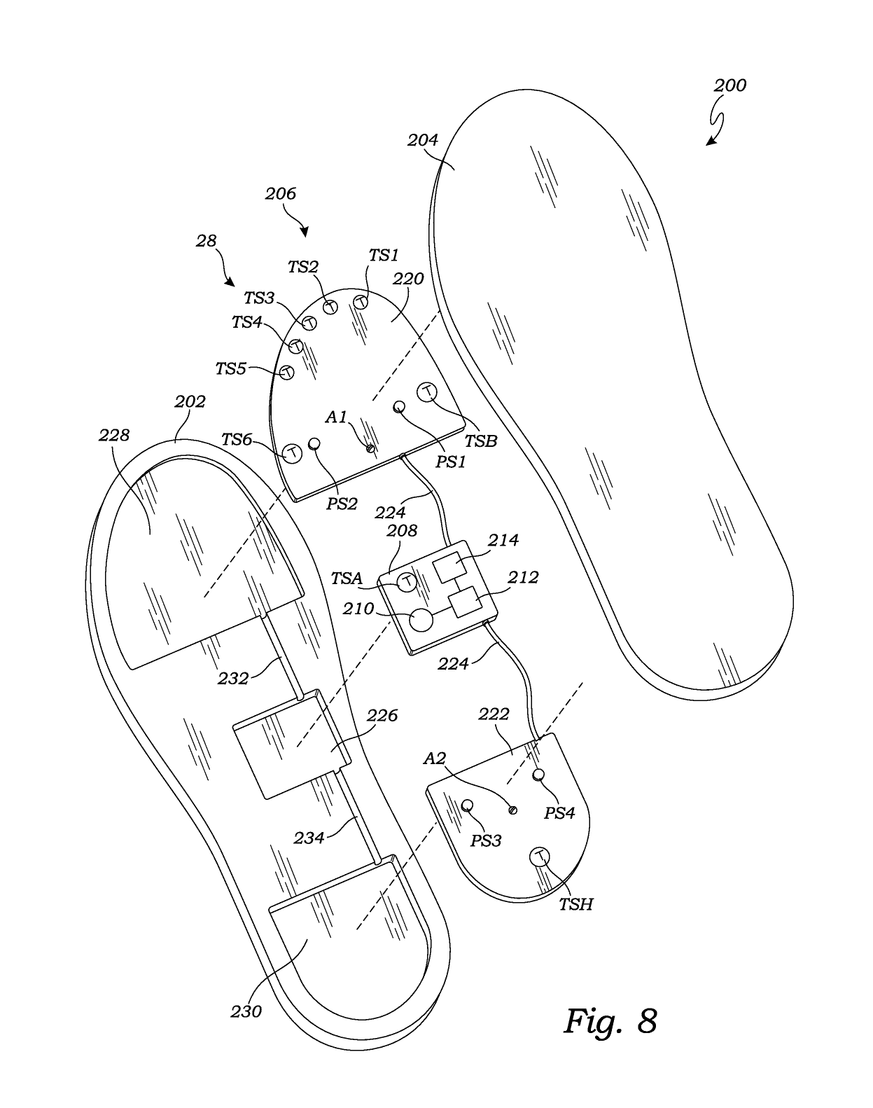

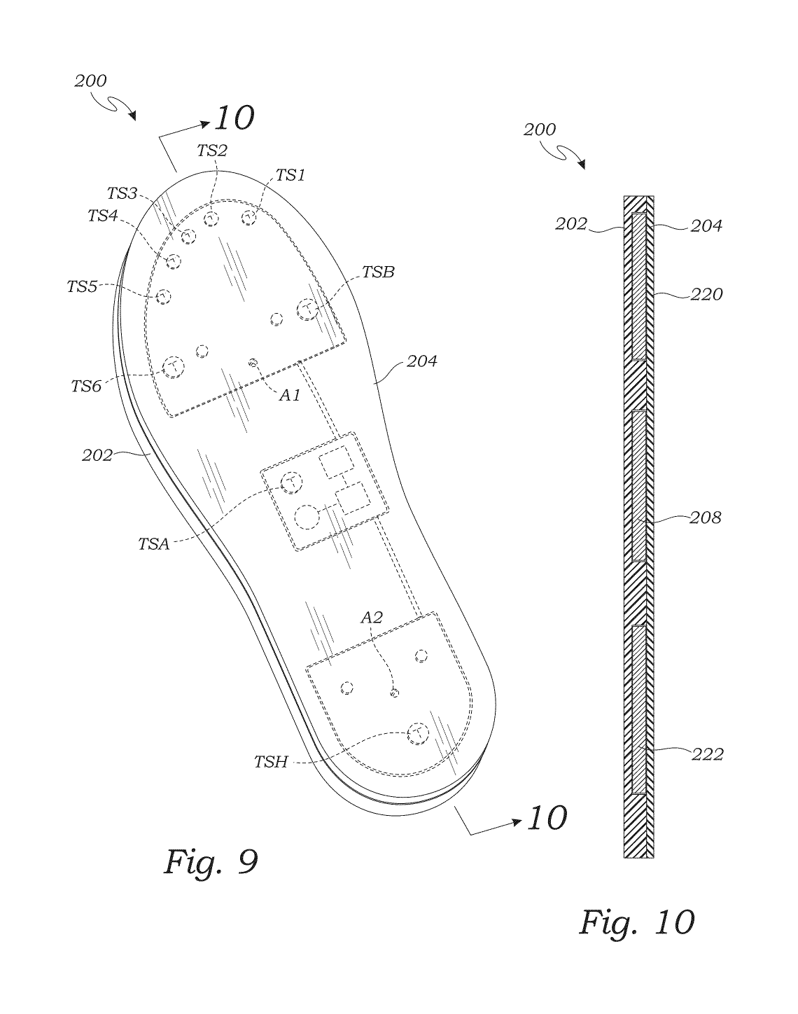

FIGS. 8-10 illustrate another embodiment of the sensor device 200. In this embodiment, the sensor device 200 (i.e, the insole described above) includes first and second layers 202 and 204 that together sandwich sensor boards 206 therebetween. In this embodiment, a central board 208 includes a battery 210, a computer processor 212, a memory 214, and a transmitter 216 (defined to include any form of transmitter, transceiver, or similar data transmission device).

In this embodiment, the sensor boards 206 further include a front sensor board 220 and a rear sensor board 222. The central board 208 of this embodiment is operably connected with front sensor board 220 and the rear sensor board 222 via flexible electrical wires 224, or other suitable connection. The front sensor board 220 is positioned so that it is under the front part (toes) of the user's foot, and the rear sensor board 222 is positioned so that it is under the rear part (heel) of the user's foot.

In the embodiment of FIGS. 8-10, the first layer 202 includes an arch recess 226, a toe recess 228, and a heel recess 230. The central board 208 is mounted in the arch recess 226. The arch recess 226 is positioned under the arch of the user's foot, so that the central board 208 is protected from impacts of the user's foot, which are more powerful at the ball and the heel of the user's foot. The front sensor board 220 is positioned in the toe recess 228 so that the temperature sensors TS1-TS5 are operably positioned beneath the user's toes. The rear sensor board 222 is positioned in the heel recess 230 so that the temperature sensor TSH is positioned under the user's heel. The central board 208 may further include the arch temperature sensor TSA, as discussed above, for sensing temperature of the user's arch.

The first layer 202 may further include conduit grooves 232 and 234 connecting the arch recess 226 with the toe recess 228, and the arch recess 226 with the heel recess 230, for receiving electrical wires 224. Alternatively, other forms of electrical connections may be provided, or the entire unit could be provided on a single board, depending upon the needs of one skilled in the art.

FIG. 11 is a perspective view of an inductive charging station 240 used to charge the insoles 20. As illustrated in FIG. 11, the inductive charging station 240 works in conjunction with an inductive charging device 242 that is operatively installed on each of the insoles 20 and operatively connected for charging the battery 26 (as illustrated in FIG. 1). The inductive charging station 240 includes a base 244 that includes an electrical cord 246 that includes a plug 248 for plugging into an external power socket (not shown). The base 244 may include any form of necessary transformers (not shown) or other electronics required.

An inductive charging mechanism 250 is operatively mounted on or in the base 244. The insoles 20, including any form of footwear that contain the insoles 20, are positioned on the base 244 so that the inductive charging mechanism 250 is adjacent the inductive charging device 242 of the insole 20, operatively recharging the insole 20 via induction, according to electromagnetic properties known in the art.

FIG. 12 is a block diagram an alternative embodiment of the sensor system 260, wherein the insoles 20 communicate with a portable electronic device 262. In one embodiment, the portable electronic device 262 may be a cell phone, tablet computer, or other similar form of portable device, with the necessary functionality being provided by a downloadable software application (commonly called an “app”). As illustrated in FIG. 12, the portable electronic device 262 includes a computer processor 264 and a memory 266, as described above. The memory 266 includes an application 268 (known as an “app”), which may be downloaded using systems known in the art. The memory 266 may also include a database 269 for storing data (although alternatively the data may merely be uploaded and not stored locally). The application 268 controls the function of the insoles 20, receives the requested data, and transmits the data via a transceiver 270 as required. The application 268 may further provide feedback to the user, via a display 272 or other mechanism. Controls 274 (e.g., touch screen, keyboard, etc.) enable the user to operatively control the portable electronic device 262.

The insoles 20 may communicate directly with a portable electronic device 262 via Bluetooth® or similar protocol. The transceiver 270 of the portable electronic device 262 may further communicate with the data collection system 110, which is discussed above, via the network 104, via a local network device 102, or alternatively via a cell network 276 or other suitable network, either a public or a proprietary system.

As illustrated in FIGS. 1-12, in use, the sensor system 10 described above, in any of the described embodiments, is used to track the foot of the user. While the system 10 may be and usually will be worn on both feet, for simplicity we will just discuss the use on a single foot. The system 10 is capable of measuring temperatures and movements of the foot. In particular, the sensor system 10 is useful for monitoring the foot of a diabetic patient, to prevent ulcers that could potentially lead to serious infections and even amputation, and also to detect restricted blood flow that can lead to damage to the foot and potential amputation.

Key to preventing ulcers is to discover infections quickly for immediate treatment, and it is often difficult to catch them in time using conventional treatments. In one method of treatment, the sensor device 20 is worn so that the temperature sensor array 50 contacts the user's toes and other critical points so that each can be monitored for increases in temperature that might warn of an impending ulcer. Information is collected by the data collection system 110, for monitoring by doctors, nurses, the user, or by a computer programmed to track the data and report potential warning signs.

The sensor device 20 also is capable of detecting drops in temperature that might indicate restricted blood flow in that portion of the foot. This potential problem can also be quickly treated if caught early.

In one embodiment, the sensor device 10 is worn all day, for constant monitoring. In another embodiment, specific measurements are taken at a predetermined time of day (or times of day), and changes in temperature over time are analyzed to determine if an ulcer is forming. The particular time frame, and the number of measurements taken, can vary depending upon the judgment of the treating doctor or other professional.

In this embodiment, at least one suitable time of day is pre-determined to record data from the temperature sensors when the temperature of the foot will be substantially unaffected by outside variables. The first thing in the morning, when the user first gets out of bed, is one such potentially suitable time, either at the same time each day, or simply whenever the user wakes up. At this time, the user should be a fairly consistent temperature, and unaffected by the environment, and/or activities. Just before bed may also be suitable, or the user may be instructed to select a particular time or times of day, and perform preparatory steps so that the temperature measurements are the most consistent (e.g., resting, bathing, wearing protective and thermally protected footwear, etc.).

At the predetermined time, the sensor system 10 is mounted on the foot, such that the temperature sensors 28 operably abut the foot for sensing the temperature of the foot. For purposes of this application, the term “abut” includes any suitable connection that transmits enough temperature for a useful reading. This can be direct contact, contact through a padded layer, contact through a sock, or any other suitable arrangement.

The temperature of the foot is then determined using the temperature sensor array 50, and the data is transmitted to the data collection system 30. These steps are repeated for a period of time, at the predetermined time, to generate a temperature trend that is substantially unaffected by outside variables. From this temperature trend it is possible to determine if an ulcer is forming. For example, if the temperature remains fairly constant for a period of time, and then suddenly spikes, it is likely that an ulcer is forming.

For example, upon determining that there is a spike in temperature, the sensor system 10 transmits a warning of potential infection. The warning may be in any form known in the art, either directly to the user (e.g., illuminating the LEDs, sounding an alert, sending the user a text or email, etc.), or to a treating professional. Similarly, upon determining that there is a drop in temperature, the sensor system transmits a warning of potential restricted blood flow to the foot.

Using this system 10 removes many false readings that may be generated by environmental factors. For example, if the user has been out walking, and the day in particularly hot or cold, and/or if the user has been wearing hot/cold footwear, these factors may skew the readings on this day. Also, if the user has been particularly active or sedentary on a given day, this may skew the readings. However, data collected under controlled conditions, and potentially at predetermined times, is particularly free of such fluctuations, and provides trend data that is accurate in detecting ulcers and other problems.

The computer or computers used in the sensor system 10 may be any form of computers or computers, servers, or networks known in the art. As used in this application, the terms computer, processor, memory, and other computer related components, are hereby expressly defined to include any arrangement of computer(s), processor (s), memory device or devices, and/or computer components, either as a single unit or operably connected and/or networked across multiple computers (or distributed computer components), to perform the functions described herein.

As used in this application, the words “a,” “an,” and “one” are defined to include one or more of the referenced item unless specifically stated otherwise. Also, the terms “have,” “include,” “contain,” and similar terms are defined to mean “comprising” unless specifically stated otherwise. Furthermore, the terminology used in the specification provided above is hereby defined to include similar and/or equivalent terms, and/or alternative embodiments that would be considered obvious to one skilled in the art given the teachings of the present patent application.

What is claimed is:

1. A sensor system for monitoring a foot, the foot having an underside that includes a heel portion, an arch portion, a ball portion, and toe portions, the sensor system comprising:
a substrate layer shaped to be worn adjacent the underside of the foot;
a temperature sensor array that includes a plurality of temperature sensors disposed on the substrate layer, including toe temperature sensors disposed on the substrate layer to correspond with each of the toe portions, and a ball temperature sensor disposed on the substrate to correspond with the ball portion of the foot;
a microcontroller operably connected with the temperature sensor array and an antenna for transmitting data received from the temperature sensor array indicating the temperatures sensed by the temperature sensor array;
a data collection system having a processor and a memory for receiving the data transmitted from the microcontroller;
a database operably installed in the memory of the data collection system for storing the data; and
a user interface program operably installed in the memory of the data collection system for displaying the data in a format suitable for determining if there is a problem with the foot based upon changes in the temperature of the foot.

2. The sensor system of claim 1, wherein the temperature sensor array includes a plurality of resistive thermal devices operably mounted on the substrate.

3. The sensor system of claim 1, wherein the temperature sensor array includes a plurality of thin film resistive thermal devices operably mounted on the substrate.

4. The sensor system of claim 1, wherein the temperature sensor array includes a plurality of thermocouples operably mounted on the substrate.

5. The sensor system of claim 1, wherein the substrate is operably mounted in a slipper having an upper for operably mounting the substrate on the foot.

6. The sensor system of claim 5, wherein the substrate is operably mounted between an inner layer and an outer layer of the slipper.

7. The sensor system of claim 1, wherein the substrate includes a first layer and a second layer, and wherein the temperature sensors are operably mounted on sensor boards that are positioned in a tow recess and a heel recess within the first layer.

8. A method for monitoring a user's foot to determine if an ulcer is forming in the foot, the method comprising the steps of:
providing a sensor system comprising:
a substrate layer shaped to be worn adjacent the underside of the foot;
a temperature sensor array that includes a plurality of temperature sensors disposed on the substrate layer;
a microcontroller operably connected with the temperature sensor array and an antenna for transmitting data;
a data collection system having a processor and a memory for receiving the data transmitted from the microcontroller; and
a database operably installed in the memory of the data collection system for storing the data;
predetermining at least one suitable time of day to record data from the temperature sensors when the temperature of the foot will be substantially unaffected by outside variables;
mounting the sensor system on the foot, at the predetermined time of day, such that the temperature sensors operably abut the foot for sensing the temperature of the foot;
determining the temperature of the foot using the temperature sensor array;
transmitting data regarding the temperature to the data collection system;
repeating these steps for a period of time at the predetermined time, to generate a temperature trend that is substantially unaffected by outside variables;
determining, based upon changes in the temperature of the foot in the temperature trend, if there is a problem with the foot.

9. The method of claim 8, wherein, upon determining that there is a spike in temperature, the sensor system transmits a warning of potential infection.

10. The method of claim 8, wherein, upon determining that there is a drop in temperature, the sensor system transmits a warning of potential restricted blood flow to the foot.

11. A method for monitoring a user's foot to determine if an ulcer is forming in the foot, the method comprising the steps of:
providing an inductive charging station that includes an inductive charging mechanism operably mounted on a base;
providing a sensor system comprising:
a substrate layer shaped to be worn adjacent the underside of the foot;
a temperature sensor array that includes a plurality of temperature sensors disposed on the substrate layer;
a microcontroller operably connected with the temperature sensor array and an antenna for transmitting data;
a battery operably connected with the microcontroller for powering the sensor system;
an inductive charging device operably connected with the battery for recharging the battery when operably coupled with the inductive charging station;
a data collection system having a processor and a memory for receiving the data transmitted from the microcontroller; and
a database operably installed in the memory of the data collection system for storing the data;
predetermining at least one suitable time of day to record data from the temperature sensors when the temperature of the foot will be substantially unaffected by outside variables;
mounting the sensor system on the foot, at the predetermined time of day, such that the temperature sensors operably abut the foot for sensing the temperature of the foot;
determining the temperature of the foot using the temperature sensor array;
transmitting data regarding the temperature to the data collection system;
repeating these steps for a period of time at the predetermined time, to generate a temperature trend that is substantially unaffected by outside variables;
determining, based upon changes in the temperature of the foot in the temperature trend, if there is a problem with the foot; and
storing the sensor system on the inductive charging station between uses, so that the battery of the sensor system is kept in a charged state via the inductive interaction between of the inductive charging device via the inductive charging mechanism.
